(19) United States
(12) Patent Application Publication
(10) Pub. No.: US 2014/0154711 A1
(43) Pub. Date: Jun. 5, 2014

(54) DETECTION OF DEFENSINS TO DIAGNOSE CANCER

(71) Applicant: ASCENDANT DIAGNOSTICS, Fayetteville, AR (US)

(72) Inventors: Anna Daily, Fayetteville, AR (US); Rohana Liyanage, Fayetteville, AR (US); Jackson O. Lay, West Fork, AR (US)

(73) Assignees: THE BOARD OF TRUSTEES OF THE UNIVERSITY OF ARKANSAS, Little Rock, AR (US); ASCENDANT DIAGNOSTICS, Fayetteville, AR (US)

(21) Appl. No.: 14/095,472

(22) Filed: Dec. 3, 2013

Related U.S. Application Data
(60) Provisional application No. 61/732,694, filed on Dec. 3, 2012.

(51) Int. Cl. G01N 33/574

(52) U.S. Cl. CPC G01N 33/574
USPC 435/7.92

(57) ABSTRACT

Non-invasive methods of detecting and providing an early diagnosis of cancer, in particular breast cancer are provided herein. The methods include obtaining a sample, suitably an ocular wash sample, from a subject and determining the level of α-Defensin 1, α-Defensin 2 and/or α-Defensin 3 in the sample. Increased levels of any of these proteins as compared to a negative control or non-cancerous subject are indicative of cancer in the subject from which the sample was obtained. Kits for performing the methods described herein are also provided.

### CROSS-REFERENCE TO RELATED APPLICATIONS

This patent application claims the benefit of priority of U.S. Provisional Patent Application No. 61/732,694, filed Dec. 3, 2012, which is incorporated herein by reference in its entirety.

### SEQUENCE LISTING

This application is being filed electronically via EFS-Web and includes an electronically submitted Sequence Listing in .txt format. The .txt file contains a sequence listing entitled “2013-12-03_{—}5945-00003_Sequence_Listing_ST25_as_filed.txt” created on Dec. 3, 2013 and is 1,230 bytes in size. The Sequence Listing contained in this .txt file is part of the specification and is hereby incorporated by reference herein in its entirety.

### INTRODUCTION

Defensins, also called Human Neutrophil Peptides (HNP's), are a family of 3-4 kDa, cationic, antimicrobial peptides, which function as part of the innate immune system expressed on numerous mucosal surfaces (Ouellette, 1999). These peptides are typically found in the granules of mature phagocytic cells, where they are stored in their pro-peptide form prior to release. The defensin family is made up of two sub-classes, α and β defensins, and classification is based on the location of cysteine residues within their sequence (Bevins, 1999).

Within the α-defensin subclass, α-defensins 1-3 have anti-tumorigenic activity. These three peptides are produced by enzymatic cleavage of a pro-peptide resulting in three peptides that differ by one amino acid each. It remains unclear whether the peptides are released by the tumor cells themselves, or released by the tumor fighting eosinophils and neutrophils responding to the effected tissue (Kemik, 2013). The presence of α-defensins 1-3 has been detected in tissue samples from colon cancer, bladder cancer, and oral cancers (Vlahou, 2001; Mizukawa, 1998; and Kemik, 2013). In addition these peptides have been observed in tissue samples from breast biopsies in response to neoadjuvant treatment and as a marker of increased probability of recovery (Bauer, 2010).

Breast cancer is a very complicated disease, which manifests in several forms within the breast duct and surrounding tissues. In addition, density of breast tissue differs significantly patient-to-patient making accurate imaging a challenge. This is an important issue as one out of every eight women are diagnosed with breast cancer over the course of their lifetime and survivability is much higher when the cancer is detected in the early stages, i.e. prior to lymph node involvement and metastasis. Currently, suspicious areas detected in a mammogram screening are then evaluated by a diagnostic mammogram, biopsy, and possibly MRI. This is a costly and time intensive procedure during which the patient is subjected to significant mental distress. One answer to this problem could lie within the proteomic profile of subjects with breast cancer. It is hoped that biomarkers detected within easily obtained bodily fluids could provide a faster, more sensitive method for determining the status of a suspicious area on a mammogram or even as a screening tool for breast cancer itself.

The single most effective way to decrease deaths associated with advanced stages of breast cancer, as well as to reduce rising health care costs, is early detection. There is unquestionably a critical need for a low cost, non-invasive screening tool to reliably increase detection of breast cancer tumors at early stages.

### SUMMARY

Non-invasive methods for screening samples, in particular ocular wash samples from a subject to detect or diagnose cancer are provided herein. The methods include obtaining a sample from the subject and performing steps for detecting the level of at least one of α-Defensin 1, α-Defensin 2 or α-Defensin 3 in the sample. If the levels of α-Defensin 1, α-Defensin 2 or α-Defensin 3 are increased as compared to the levels in a control sample the subject is likely to have cancer.

in another aspect, the methods include obtaining a sample from a subject and preparing the sample for liquid chromatography-mass spectroscopy. The liquid chromatography-mass spectroscopy spectra of the sample is collected and peaks with increased intensity corresponding to at least one of α-Defensin 1, α-Defensin 2 or α-Defensin 3 are identified in the sample. It can then be determined from the spectra whether the subject is at increased risk of having cancer if the subject has a peak with increased intensity corresponding to any one of α-Defensin 1, α-Defensin 2 or α-Defensin 3 as compared to the levels in a control sample.

In yet another aspect, the methods include obtaining a sample from the subject; and performing an antibody based detection assay on the sample to detect the level of at least one of α-Defensin 1, α-Defensin 2 or α-Defensin 3 in the sample. If the levels of α-Defensin 1, α-Defensin 2 or α-Defensin 3 are increased as compared to the levels in a control sample the subject likely has cancer.

In still another aspect, kits for detecting cancer in a subject including an antibody capable of detecting at least one of α-Defensin 1, α-Defensin 2 or α-Defensin 3 and instructions for performing the assay are provided.

### BRIEF DESCRIPTION OF THE DRAWINGS

FIG. 1 is a set of figures showing the MALDI-TOF spectra collected from breast cancer OW (ocular wash) samples, benign OW samples, and control OW samples. Samples were analyzed for significant spectral differences using ClinProTools2 software. In FIG. 1A a region of interest was observed between 3350 and 3500 Da that contained three peaks with significant intensity increase with respect to breast cancer OW samples. FIG. 1B shows further analysis of spectral intensity using the gel view feature within ClinProTools and further validates this region as an area of interest by three “bands” with significant intensity indicated by darker regions which correspond to the arbitrary units on the y-axis. FIG. 1C is a graph showing the intensity distribution between control OW samples (class 1), breast cancer OW samples (class 2) and benign OW samples (class 3) and also indicates a significant increase in intensity for class 2 samples further validating this as a region of interest.

FIG. 2 shows liquid chromatography-mass spectroscopy (LC-MS) spectra comparing three sample pools: breast cancer pooled OW samples (medium gray) benign pooled OW samples (dark gray) and control pooled OW samples (light gray). Peaks at 40-42 min are significantly up regulated with respect to the breast cancer OW pool. Molecular weights determined by extracted ion chromatograms of these peaks are identical to the three masses detected in the MALDI-TOF spectra. The peak eluting at 40 min contained a mass of 3441 Da while the peak eluting at 42 min contained masses of 3370 Da and 3485 Da.

FIG. 3 shows the MALDI-TOF spectra of fractions generated by injection of pooled breast cancer OW samples on the LC-MS instrument. Fractions were collected every minute from 1-100 minutes, however, since importance was placed on time points 40-42 min, only fractions eluted from 35-50 minutes were retained for further testing. A total of four 100 μL injections were carried out and the volume of each of the retained fractions was reduced from 700 μL to 20 μL prior to spotting on the MALDI target. As expected the peaks of interest eluted in fractions 40, 41, and 42, with mass 3445 eluting cleanly at 40 min, mass 3375 eluting over 41 and 42 mins, and mass 3488 eluting at 42 min.

FIG. 4 shows the MALDI-TOF spectra of fractions 40, 41, and 42 following reduction, alkylation, and trypsin digestion. Masses of generated fragments were uploaded to the MASCOT database for protein identification.

### DETAILED DESCRIPTION

Provided herein are non-invasive methods for screening samples, in particular ocular wash samples or lachrymal secretions from a subject in order to detect or diagnose cancer. Early cancer diagnosis and treatment are key to overcoming cancer, preserving the life of the patient and limiting the need for invasive treatments. The ability to treat the cancer and the cost of treatment are directly related to how early the cancer is diagnosed. For breast cancer, early diagnosis is directly related to survival and many patients either cannot afford or for other reasons do not get regular mammography to allow early detection of breast cancer. Thus, methods of detecting breast cancer using an inexpensive, non-invasive assay are needed.

The methods provided herein include obtaining a sample from the subject and detecting the level of at least one of α-Defensin 1, α-Defensin 2 or α-Defensin 3 in the sample. Suitably at least two or all three of the Defensins are assessed in the method. If the levels of α-Defensin 1, α-Defensin 2 or α-Defensin 3 are increased as compared to the levels in a control sample the subject is likely to have cancer. In particular the sample may be an eye wash or lachrymal secretion sample.

As demonstrated in the Examples, human tear fluid contains proteins that either individually as biomarkers or collectively as a biosignature can be used as a diagnostic indicator of breast cancer or other cancers. In particular early stage breast cancers can be detected using a non-invasive procedure. Additionally, the use of tears or ocular wash samples in the analysis of proteins useful as biomarkers in breast cancer detection has several advantages. First, the collection of tears for the analysis is relatively easy and non-invasive. Secondly, mass spectrometry or an antibody-based assay, such as an ELISA, can be performed directly on the sample and requires limited sample separation or processing when compared to requirements of other biological samples such as blood serum or other tissue fluids.

The Examples compare the protein profile as determined by LC-MS in ocular wash fluid from breast cancer patients with those without breast cancer or benign samples for differences that identify certain proteins as diagnostic indicators of the disease. The present invention also contemplates evaluating the usefulness of proteins identified or protein patterns (signatures) found in tears besides their use in diagnosis of cancer. The Examples demonstrate the usefulness of determining the levels of α-Defensin 1, α-Defensin 2 and/or α-Defensin 3 in tear fluid. Increased levels of any one of α-Defensin 1, α-Defensin 2 or α-Defensin 3 are indicative of breast cancer in the subject. Elevated levels of these proteins may also be indicative of other cancers and their elevation may not be limited to ocular wash samples, but may also be found in other bodily fluids such as saliva, blood or serum. In addition to developing a screening tool for the detection of breast cancer, tears and the levels of the α-Defensin 1, α-Defensin 2 or α-Defensin 3 proteins may be used to assess treatment response of breast cancer or to ensure that a treated cancer remains in remission. Further, it is also contemplated that this screening technique could be used in the future as a point of service assay or even as an over-the counter diagnostic test similar to currently available pregnancy tests. It is contemplated that a small amount of lachrymal fluid placed on test paper can be compared with appropriate controls to enable a subject to obtain a diagnostic result at home with great ease. Such a screening assay may allow detection of cancers earlier. For example, some of the cancers detected in the Examples were stage II cancers. It is contemplated that the assays provided herein could be used to detect stage I or stage II cancers, in particular breast cancers.

The subject is suitably a mammal, more suitably a human. The subject may be a healthy individual, an individual suspected of having cancer, suitably breast cancer, or an individual at high risk of developing cancer, i.e. an individual with a genetic predisposition for developing the cancer. The individual may have been diagnosed with cancer and the cancer may have been previously treated and be in remission or undergoing cancer treatment. The subject may have a palpable lump suspected of being cancerous and the assay described herein may be used as a non-invasive procedure to evaluate the lump. The sample and the control sample may be any biological sample, but it is suitably an ocular wash or tear fluid. The control sample may be obtained from a healthy individual or may be a composite sample from a pool of healthy donors. It is contemplated that such a control sample may not be a biological sample, but may be made using known concentrations of the proteins based on the amount of the proteins generally found in healthy individuals, i.e. a positive and/or negative control. Specifically, the diagnostic indicators detected by this method are useful for detecting breast cancer. More specifically, α-Defensin 1, α-Defensin 2 and α-Defensin 3 are shown to be up-regulated in individuals having cancer.

Methods of screening an individual for cancer, comprising: obtaining a biological sample from the individual; determining the amount(s) of α-Defensin 1, α-Defensin 2 and/or α-Defensin 3 in the biological sample; and comparing the amount(s) in the sample form the individual to the amount(s) of the same protein(s) in a control sample, i.e. a sample from an individual without cancer or alternative that the amount of the protein(s) in the sample are above a threshold known to represent the amount of the protein(s) in samples from individuals with the cancer. Increased amount(s) of α-Defensin 1, α-Defensin 2 and/or α-Defensin 3 in the biological sample compared to the negative control sample or above the threshold indicates that the individual has cancer.

In the Examples, the three proteins were separated by LC-MS and their amino acid sequences were identified. These proteins have characteristic MS spectra and thus may be identified based on their spectral peaks alone without the need to identify the amino acid sequences of the proteins. The intensity of the peaks is related to the level of expression and thus the samples can be compared to the controls based on the intensity of the peaks. The MS profile obtained from a subject can then be compared with the MS protein fingerprint profile for breast cancer and/or for a healthy subject. A match of the test profile with the control fingerprint profile indicates that the test individual has breast cancer. Thus this method eliminates the additional step of having to quantify protein markers or identify the proteins to diagnose a diseased state. Signature profiles obtained by MS are very specific for a given biological sample. Such profiles because of their high specificity have been used previously to identify different species of bacteria. As shown in the Examples, the LC-MS peaks corresponding to α-Defensin 1, α-Defensin 2 or α-Defensin 3 are found at 40, 41 and 42 minutes and correspond to peaks at 3375 Daltons, 3445 Daltons or 3488 Daltons. The levels of each Defensin may be increased relative to a control sample from a healthy donor by at least 2 fold, 3 fold, 5 fold, 7 fold or even 10 or more fold.

Alternatively, an antibody-based detection assay may be performed to detect the level of α-Defensin 1, α-Defensin 2 and/or α-Defensin 3 in the sample. Antibodies to α-Defensin 1, α-Defensin 2 and α-Defensin 3 are commercially available and additional antibodies to these proteins can be developed using methods available to those of skill in the art. Antibody-based detection assays include enzyme-linked immunosorbent assay (ELISA), Western blot or immunohistochemistry assays. Additionally, the amount of protein can be determined by using other immunoassays that are known in the art such as radioimmunoassay and agglutination inhibition reactions. An ELISA assay may be specific for one or more of α-Defensin 1, α-Defensin 2 and/or α-Defensin 3 in the sample. In one embodiment, all three α-Defensins are increased in a sample from a subject with cancer. The ELISA assay may be performed as a rapid assay using a test strip and may provide a simple colorimetric read-out. Suitable assays may provide a positive-negative result or may provide qualitative information regarding the levels of α-Defensin 1, α-Defensin 2 and/or α-Defensin 3 in the sample.

This method further comprises assessing a response to a treatment for cancer in an individual. This assessment step comprises: comparing the amount(s) of α-Defensin 1, α-Defensin 2 and/or α-Defensin 3 in the sample from the individual collected post-treatment with the amount(s) of the protein(s) in a control sample or in the sample of the individual collected pre-treatment. If the amount(s) of the protein(s) collected post-treatment is the same as the amount(s) of the protein(s) in the control sample or decreased compared to the amount(s) of the protein(s) collected pre-treatment it is indicative that the individual is responsive to the treatment. If α-Defensin 1, α-Defensin 2 and/or α-Defensin 3 are at the same level or increased after treatment, it is indicative that treatment is not effective.

All other aspects regarding the type of biological and control samples and cancer is the same as described above. In general, samples of individuals either suspected of having cancer, at risk of developing cancer, being treated for cancer or as a follow-up after treatment to monitor for recurrence can be screened using the method described above. Individuals suspected of having cancer or at risk of developing cancer are identified based on clinical symptoms such as an abnormal mammogram/ultrasound or a palpable lump or a benign breast evaluation, genetic predisposition and/or lifestyle.

Further, kits for screening samples, in particular tears, ocular washes or other lachrymal secretions for cancer, including but not limited to breast cancer, are provided herein. The kits may be based on an ELISA based assay and may produce a colorimetric result. These kits can be used with the same ease as the commonly available over the counter pregnancy test kit. The kits may be used in a doctor's office or may provide a home screen test. The kits will include an antibody capable of binding to and allowing detection of at least one of α-Defensin 1, α-Defensin 2 or α-Defensin 3 and instructions for use of the kit and interpretation of the results.

This kit may also include α-Defensin 1, α-Defensin 2 or α-Defensin 3 as positive controls, an antibody to protein(s) identified as positive control proteins found in samples from both cancer free and cancer samples, reagents to detect the binding of the antibody to the protein and eye wash collection material. Preferably, the biological sample that is tested with this kit is a tear sample or an ocular wash sample. The container means of the kits will generally include at least one vial, test tube, flask, bottle, or even syringe or other container means, into which the antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

The following examples are meant only to be illustrative and are not meant to limit the scope of the invention or of the appended claims. All references cited herein are hereby incorporated by reference in their entireties.

### EXAMPLES

### Materials

ZipTip_{C18} were purchased from Millipore. Acetonitrile (ACN), methanol, and trifluoracetic acid (TFA) were purchased from VWR International, LLC. MALDI matrices, α-cyano-4-hydroxycinnaminic acid (HCCA), sinapinic acid, and 1,5 dihydroxybenzoic acid (DHB) were purchased from Bruker Daltonics of Billerica, Mass. Protein calibration standard I and Peptide calibration standard II were also purchased from Burker Daltonics. Optics laboratory Eye-Cept Rewetting drops, single use droppers, were purchased from www.drugstore.com as well as www.amazon.com. One cc syringes and 0.5 mL tubes were purchased from VWR international, LLC. Dithiothreitol and iodoacetamide were purchased from Sigma Aldrich. Trypsin was purchased from Promega. Ammonium Bicarbonate was purchased from VWR international.

### Methods:

### Protocol for Collecting Ocular Wash Samples:

Ocular wash samples were collected using an institutional review board (IRB) approved procedure that involves washing of the exposed ocular surface with a preservative free eye wash solution. In addition all patients enrolled in the study signed a protocol specific informed consent. An Optics Laboratory Eye-Cept Rewetting drops, single use dropper is used to apply approximately five drops of rewetting saline to the outside corner of the eye. The solution is then allowed to roll across the surface of the eye and collect in the inner corner/duct next to the nose. The solution is then collected by suction using a one mL tuberculine syringe, with no needle attached, and transferred to a pre-labeled 0.5 mL tube with an o-ring screw top cap. The total volume from each collection is approximately 100 μL. Samples collected at clinics are then stored between −20° C. and −80° C. (depending on freezer unit available) within two hours of collection.

Control samples are collected using the procedure detailed above, from volunteers (both male and female) between the ages of 18-100 who are cancer and mass free as per the inclusion criteria outlined in the IRB approved collection protocol. All control samples are recruited from the general population. Volunteers enrolled in the study as controls also sign a protocol specific informed consent.

Samples from patients in treatment for breast cancer are collected from clinics in Fayetteville and Rogers, Ark. Patients with breast cancer are given the opportunity to participate in the study by donation of tear samples. Research nurses at the clinics participated in a training session given by the Applicant, and are responsible for gathering patient consent, gathering patient profile questionnaires, and collecting the tear samples.

All patients at the clinic undergoing a biopsy procedure are given the opportunity to donate a sample. These samples are being collected by a team of research nurses who participated in a training session and are responsible for gaining consent, gathering patient profile questionnaires, and collecting the tear samples. Samples which come back from pathology assessment as negative for breast cancer comprise the benign mass group for data comparison.

All clinic samples are retrieved on a weekly basis and transferred on dry ice to the laboratory facility. Three 15 μL aliquots are made for each sample, then all aliquots and any remaining sample volume are stored at −80° C. until future use. Aliquoting helps prevents the main sample from being thawed and refrozen for each test that may be needed.

### Ocular Wash Sample Purification for MALDI:

Prior to MALDI testing, tear samples were purified using ZipTip_{c18}. This procedure serves to remove any contaminates that may be present in the sample and to concentrate the proteins in order to increase ease of detection. A 15 μL aliquot was removed from the freezer and thawed at room temperature for 10 minutes (−22° C.). The protocol for ZipTip_{c18} was adapted from the user manual supplied by Millipore and a variable pipette with a total volume capacity of 10 μL was used for all sample preparations. The ZipTip_{c18} was equilibrated in a wetting solution of acetonitrile (ACN) 0.1% TFA for 10 cycles (1 cycle involves aspirating 10 μL of solution into the tip and dispensing). Following equilibration, the tip was washed with ddH₂O (0.1% TFA) for 10 cycles. The sample was then loaded for 10 cycles, followed by a wash with ddH₂O (0.1% TFA) for 10 cycles. The load procedure, followed by the wash procedure was carried out a total of five times to ensure maximum protein binding. Bound proteins were eluted in 5 μL of ACN (0.1% TFA) for 20 cycles into a clean tube. The ACN (0.1% TFA) was removed using an eppendorf vacufuge plus for 10 minutes at 45° C. Samples were then reconstituted in 5 μL, ddH₂O (0.1% TFA) and spotted onto a ground steel MALDI target. Each sample was spotted a total of three times at 1 μL each time, allowing complete drying of the spot before more material was added. After the final spotting was completely dry, 1 μL of a saturated solution of 40 mgs of Sinapinic Acid matrix prepared in 1 mL of 50:50 solution of ACN/ddH₂O (0.1% TFA) was spotted onto each sample and all samples were allowed to dry completely on the bench top prior to data collection. One microliter of protein standard was added to several locations on the MALDI target as well. The protein standard was spotted only once and followed by addition of the sinapinic acid matrix used for the OW samples.

### MALDI Data Acquisition

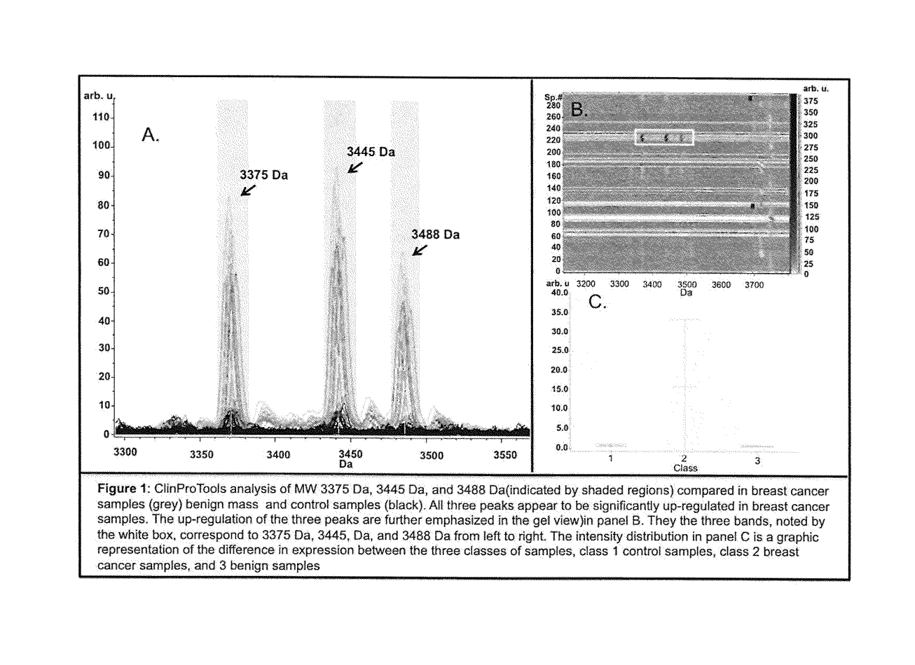

Data was collected on a Bruker Reflex III MALDI-TOF mass spectrometer in its linear positive mode, as linear mode increases the sensitivity. Acquisition of all spectra was performed both manually and automatically (user unbiased acquisition) using Bruker Daltonics flex Control software. For each spot, MALDI-TOF mass spectra were acquired at least three times, with a total of 200 laser shots accumulated for each run. Shot accumulation was programmed using a fuzzy logic operator to only consider spectra with S/N better than 20 in between m/z 2000-45,000. Peaks of interest were identified within the MALDI spectrum at 3375 Da, 3445 Da, and 3488 Da. Individual spectra from 20 cancer samples, 20 benign samples, and ten control samples were uploaded into the ClinProTools software package from Bruker to identify spectral regions with significant intensity differences between the three peaks among breast cancer, benign and control OW samples (FIG. 1).

MALDI-TOF data was collected using automatic data collection procedures on breast cancer OW samples (ten total), benign OW samples (ten total) and control OW samples (ten total). After the spectra were baseline corrected the data was uploaded to Burker's ClinProTools software to determine if any biomarkers may be present. FIG. 1 indicates an area between 3350-3500 Da where three peaks show significant up-regulation with respect to breast cancer. Grey peaks further defined by the shaded regions are from breast cancer samples. Data in black is from benign and control samples.

To ensure the increased intensity was specific to the three peaks of interest, the entire MALDI-TOF spectra was analyzed to be sure that increases in intensity were not occurring for all peaks from breast cancer OW samples. In order to confirm these potential biomarkers, a larger sample pool was analyzed for a total of 75 breast cancer OW samples, 24 benign OW samples, and 15 control OW samples. Preliminary sensitivity and specificity values were calculated by examining the MALDI-TOF spectra of all samples to determine if any one of the three α-defensin peptides was present. Values for all three peaks were calculated independently (Table 1).

TABLE 1
Biomarker Parameters

| Marker | Sensitivity | Specificity |
|---|---|---|
| 3375 Da | 62% | 73% |
| 3445 Da | 62% | 73% |
| 3488 Da | 49% | 80% |

### Ocular Wash Sample Preparation for LC-MS

In order to generate a baseline LC-MS profile for each class, tear samples were pooled into three groups: breast cancer OW samples, benign OW samples, and control OW samples. Each pool was 100 μL total consisting of 10 μL from 10 different samples of the appropriate type. Samples were pooled to increase detection sensitivity of the peaks by the LC-MS. In addition to the LC-MS data, MALDI-TOF data was also collected for each sample individually to continue the statistical analysis of the samples.

### LC-MS Data Collection

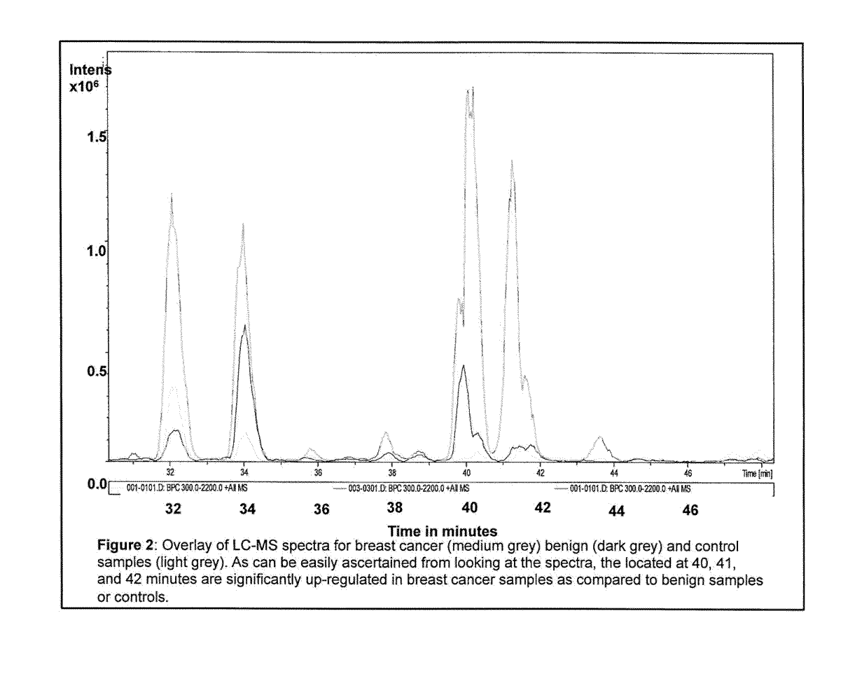

LC-MS data collection was carried out on a HP-1100 LC system with a BioRad model 2128 fraction collector and ESQUIRE-LC quadruple ion trap electrospray ionization mass spectrometer. The following gradient was used for optimal separation: time 0-5 min at 15% ACN (0.1% TFA), 5-110 min a linear gradient from 15% ACN (0.1% TFA) to 34% ACN (0.1% TFA) was carried out to ensure maximum separation of low molecular weight proteins. From 110-120 min the linear gradient went from 34% to 100% ACN (0.1% TFA) primarily to act as a cleaning step for the column. Initially, effort was placed on identifying the elution time of the three low mass peaks observed in the MALDI spectra at 3375 Da, 3445 Da, and 3488 Da. Peaks corresponding to these masses were identified to elute between 38 and 44 min. LC-MS profiles for each class were compared to evaluate the presence of a significant intensity difference as observed by MALDI-TOF. Upon overlaying the profiles from the three groups, it was evident the increased intensity due to these three peaks was not specific to MALDI-TOF data and therefore was significant. (FIG. 2).

### Determination of Protein Identity:

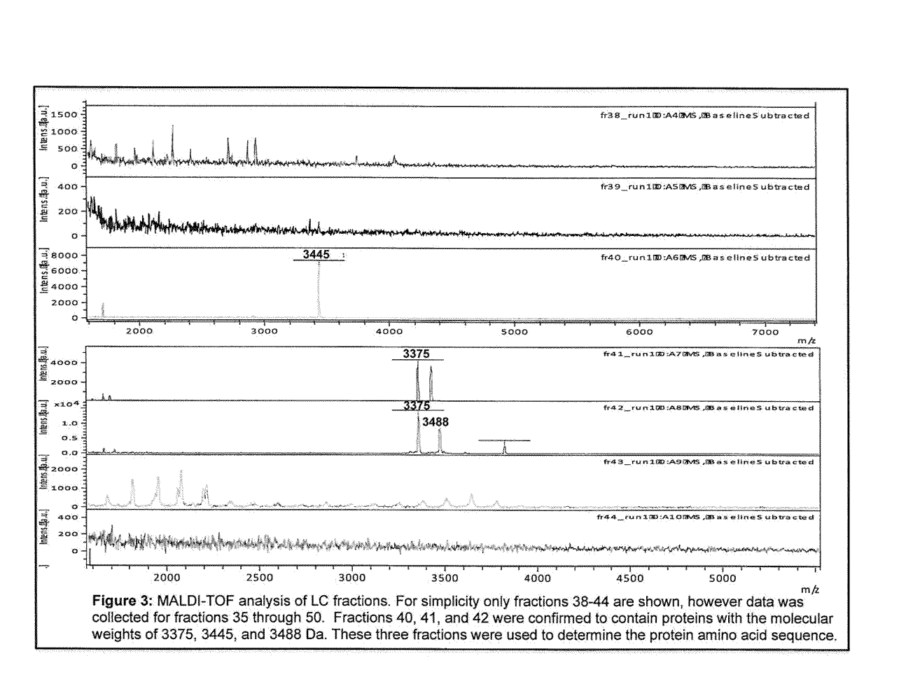

A 420 μL sample was made by pooling 30 μL from 14 previously untested breast cancer samples. A total of four injections were completed on the HP-1100 LC system, with each injection volume being 100 μL. To ensure complete collection of the proteins of interest, fractions from 35 min to 50 min were retained after each injection. Fraction volumes were reduced from 700 μL to 20 μL using an eppendorf vacufuge, and directly spotted on the MALDI target 1 μL at a time for three additions total. A solution of 20 mgs of HCCA in 50:50 ACN/ddH20 (0.1% TFA) was prepared and 1 μL was added to each spot. MALDI-TOF data was collected, using the automated procedure described above, on each of the 15 fractions from each of the four independent injections, to determine which fractions contained the peaks of interest. Fraction 40 contained peak 3437 Da (exact mass for 3445), Fraction 41 contained 3365 Da (exact mass for 3375) and fraction 42 contained both 3365 Da and 3488 Da (FIG. 3).

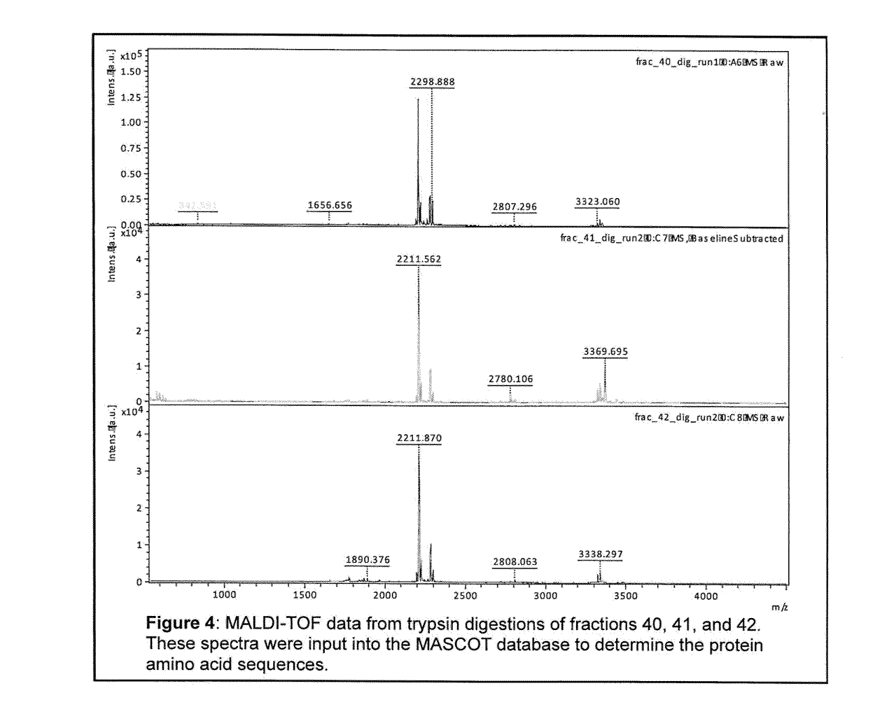

Corresponding fractions from each injection were pooled, and all solvent was removed by evaporation using the eppendorf vacufuge. Each sample (40, 41, and 42) was reconstituted in 100 μL of 50 mM Na₄Co₃. Disulfide reduction was carried out by adding 5 μL of a 200 mM DTT and boiling the sample for 10 minutes. A sulfhydryl alkylation reaction immediately followed by adding 4 μL of a 1 M iodoacetemide solution and placing the sample in the dark at room temperature for one hour. The sulfhydryl alkylation was stopped by addition of 20 μL 200 mM DTT stock. Finally each fraction underwent an 18 hour trypsin digestion at 37° C. Following digestion, a ZipTipc18 prep was carried out as described above and MALDI-TOF spectra were collected for each fraction to determine the m/z of the peptide fragments produced from trypsin digestion. (FIG. 4). The MALDI-TOF spectra of the digested fragments were up loaded to the MASCOT data base using fixed modifications of carbamidomethyl and variable modifications of oxidation.

MASCOT analysis indicated a the protein identity of the three peaks to be human α-Defensin 1, 2, and 3 for peaks 3445 Da, 3375 Da, and 3488 Da, respectively. A protein score above 66 is considered significant and each search for our biomarkers had a protein score over 92%. The protein identities are listed in Table 2. Alpha-Defensin's are widely expressed within the female reproductive tract and are produced in response to tumor markers but have not been previously shown to be up-regulated in regards to breast-cancer (Wang, 2009; and Hickey, 2012).

TABLE 2
Biomarker Identities

| Protein | Sequence | MW |
|---|---|---|
| α-Defensin 1 | ACYCRIPACIAGERRYGTCIYQGRLWAFCC | 3440 |
| α-Defensin 2 | CYCRIPACIAGERRYGTCIYQGRLWAFCC | 3365 |
| α-Defensin 3 | DCYCRIPACIAGERRYGTCIYQGRLWAFCC | 3484 |

### LC-MS CLIA Test

The first application of this technology will be to develop a CLIA based protocol utilizing a triple quadrupole LC-MS platform, which will be carried out at a centralized laboratory testing facility. Ocular wash samples will be collected in clinics using provided collection kits, and samples will then be shipped to the testing facility. Ideally, quantitative analysis of diagnostic peptides produced in the triple quad will be carried out and the information will be reported to the consulting physician. The quantitative information will be accompanied with a threshold value above which the patient's likelihood of having cancer significantly increases. Results given to the physician will state the patient's value and a test qualifier of pass/tail/unable to process

### ELISA Assay

The second phase of test development will encompass developing a point of care ELISA based assay for the detection of these protein biomarkers in tear samples. Since the protein sequences only differ by one amino acid, one antibody will be generated to detect all three α-defensin variants. In the samples that we have collected to date, an increase in the level of Defensin 1, 2, or 3 is primarily associated with breast cancer samples, therefore an increase in detection can be directly correlated with breast cancer samples. Further validation will be needed to define this technique, but with the use of commercial antibodies this assay could easily be adapted to a variety of biomarker detection instrumentation systems. Also, this technology could be adapted for a quick use cartridge or kit with color detection, similar to a pregnancy test. These applications would be very useful in areas of the world where advanced equipment is not affordable.

### REFERENCES

Ouellette A. J. (1999) Mucosal Immunity and Inflammation IV. Paneth cell antimicrobial peptides and the biology of the mucosal barrier. American Journal of Physiology. G257-G261.
Bevins et. al., (1999) Defensins and innate host defence of the gastrointestinal tract. Gut. 45:911-915.
Bauer J. A., Chakravarthy A. B., Rosenbluth J. M. et. al. (2010). Clinical Cancer Research. Identification of Markers of Taxane Sensitivity Using Proteomic and Genomic Analysis of Breast Tumors from Patients Receiving Neoadjuvant Paclitaxal and Radiation. 16:681-690.
Mizukawa N. S., Sugiyama K. Fukunaga J., Ueno T., Mishima K., Takagi, S., et al. Defensin-1 a peptide detected in the saliva of oral squamous cell carcinoma patients. (1998) Anticancer Research. 3, 410-416.
Vlahou A., Schellhammer P. F., Mendrinos S., Patel, K., Kondylis F. I., Gong L., et al. (2001) Development of a novel proteomic approach for the detection of transitional cell carcinoma of the bladder in urine. American Journal of Pathology. 4:1491-1502
Kemik O., Kemik A. S., Sumer A., Begenik H., Purisa S., Tuzun S., (2013) Human neutrophil peptides 1, 2, and 3 (HNP 103): elevated serum levels in colorectal cancer and novel marker of lymphatic and hepatic metastasis. Human & Experimental Toxicology. 32(2) 167-171.
Sprague B. L., Trentham-Dietz A., Gangnon R. E., Ramchandani R., Hampton J. M., Robert S. A., Remington P. L., and Newcomb P. A. (2011) Socioeconomic status and survival after an invasive breast cancer diagnosis, Cancer 117:1542-1551.
Warner E., Causer P. A., Wong J. W., Wright F. C., Jong R. A., Hill K. A., Messner S. J., Yaffe M. J., Narod S. A., and Plewes D. B. (2011) Improvement in DCIS Detection Rates by MRI Over Time in a High-Risk Breast Screening Study, Breast J 17: 9-17
Lufiner D., and Possinger K. (2002) Nuclear matrix proteins as biomarkers for breast cancer, Expert Rev Mol Diagn 2:23-31.
Kolb T. M., Lichy J., and Newhouse J. H. (2002) Comparison of the performance of screening mammography, physical examination, and breast US and evaluation of factors that influence them: an analysis of 27,825 patient evaluations, Radiology 225:165-175.
Galukande M., and Kiguli-Malwadde E. (2010) Rethinking breast cancer screening strategies in resource-limited settings, Afr Health Sci 10:89-92.
Lobb R., Ayanian J. Z., Allen J. D., and Emmons K. M. (2010) Stage of breast cancer at diagnosis among low-income women with access to mammography, Cancer 116: 5487-5496.
Wang F., McLafferty, S. Escamilla, V., and Luo L. (2008) Late-Stage Breast Cancer Diagnosis and Health Care Access in Illinois, Prof Geogr 60:54-69.
Huang B., Dignan, M., Han D., and Johnson O. (2009) Does distance matter? Distance to mammography facilities and stage at diagnosis of breast cancer in Kentucky, J Rural Health 25:366-371.
Henry K. A., Boscoe F. P., Johnson C. J., Goldberg D. W., Sherman R., and Cockburn M. (2011) Breast Cancer Stage at Diagnosis: Is Travel Time Important?, J Community Health.
Williams K. P., Mabiso A., Todem D., Hammad A., Hill-Ashford Y., Hamade H. Palamisono G., Robinson-Lockett M., and Zambrana R. E. (2011) Differences in knowledge of breast cancer screening among African American, Arab American, and Latina women, Prev Chronic Dis 8:A20.
Schummer M., Beatty D., and Urban N. (2010) Breast cancer genomics: normal tissue and cancer markers, Ann NY Acad Sci 1210:78-85.
Schilling K., Narayanan D., Kalinyak J. E., The J., Velasquez M. V., Kahn S., Saady M., Mahal R., and Chrystal L. (2011) Positron emission mammography in breast cancer presurgical planning: comparisons with magnetic resonance imaging, Eur J Nucl Med Mol Imaging 38:23-36.
Wang Y.-S., Li D., Shi H.-S., Wen Y.-J., Yang L., Xu N., Chen X.-C., et al. (2009). Intratumoral expression of mature human neutrophil peptide-1 mediates antitumor immunity in mice. Clinical cancer research: an official journal of the American Association for Cancer Research, 15(22):6901-6911.
Hickey D. K., John F. V., and Wira C. R. Mouse estrous cycle regulation of vaginal versus uterine cytokines, chemokines, α-/β-defensins and TLRs. (2013). Mouse estrous cycle regulation of vaginal versus uterine cytokines, chemokines, α-/β-defensins and TLRs. Innate Immunity, 19(2):121-131.

We claim:

1. A method of determining whether a subject is likely to have cancer comprising: obtaining a sample from the subject; performing steps for or detecting the level of at least one of α-Defensin 1, α-Defensin 2 or α-Defensin 3 in the sample; and determining the subject has cancer if the levels of α-Defensin 1, α-Defensin 2 or α-Defensin 3 are increased as compared to the levels in a control sample.

2. The method of claim 1, wherein the cancer is breast cancer, the subject is a human and the sample is an ocular wash sample.

3. The method of claim 1, wherein all three Defensins are increased in the sample as compared to a negative control.

4. A method of determining whether a subject is likely to have cancer comprising: obtaining a sample from the subject; preparing the sample for liquid chromatography-mass spectroscopy; collecting liquid chromatography-mass spectroscopy spectra of the sample; identifying whether the sample contains peaks with increased intensity corresponding to at least one of α-Defensin 1, α-Defensin 2 or α-Defensin 3 in the sample; and determining the subject is at increased risk of having cancer if the subject has a peak with increased intensity corresponding to any one of α-Defensin 1, α-Defensin 2 or α-Defensin 3 as compared to the levels in a control sample.

5. The method of claim 4, wherein the peaks with increased intensity are found at about 3375 Daltons, about 3445 Daltons or about 3488 Daltons.

6. The method of claim 4, wherein the sample is an ocular wash.

7. The method of claim 4, wherein the subject is human.

8. The method of claim 4, wherein the subject is suspected of having cancer.

9. The method of claim 4, wherein the subject is at increased risk for developing a cancer.

10. The method of claim 4, wherein the cancer is breast cancer.

11. The method of claim 10, wherein the subject has a palpable lump suspected of being cancerous.

12. The method of claim 4, wherein the cancer is detected as a stage II breast cancer.

13. The method of claim 4, wherein all three Defensins are increased in the sample from subjects likely to have cancer.

14. The method of claim 4, wherein the subject is likely to have cancer when the level of at least one of α-Defensin 1, α-Defensin 2 or α-Defensin 3 is increased 5 fold as compared to levels in a negative control sample.

15. A kit for detecting cancer in a subject, the kit comprising an antibody capable of detecting at least one of α-Defensin 1, α-Defensin 2 or α-Defensin 3 and instructions for performing the assay.

16. The kit of claim 15, further comprising reagents for performing an enzyme linked immunosorbent assay.

17. The kit of claim 15, further comprising controls for analyzing the results of the assay.

18. A method of determining whether a subject is likely to have cancer comprising: obtaining a sample from the subject; performing an antibody based detection assay on the sample to detect the presence and/or level of at least one of α-Defensin 1, α-Defensin 2 or α-Defensin 3 in the sample; and determining the subject is likely to have cancer if the levels of α-Defensin 1, α-Defensin 2 or α-Defensin 3 are increased as compared to the levels in a control sample.

19. The method of claim 18, wherein the antibody based detection assay is an enzyme linked immunosorbent assay using an antibody specific for at least one of α-Defensin 1, α-Defensin 2 or α-Defensin 3.

20. The method of claim 19, wherein the enzyme linked immunosorbent assay is performed as a rapid assay using a test strip.

21. The method of claim 19, wherein the enzyme linked immunosorbent assay provides a colorimetric concentration dependent read-out.


SEQUENCE LISTING (3 sequences)

SEQ ID NO: 1 - protein, 30 aa, homo sapiens
ACYCRIPACIAGERRYGTCIYQGRLWAFCC

SEQ ID NO: 2 - protein, 29 aa, homo sapiens
CYCRIPACIAGERRYGTCIYQGRLWAFCC

SEQ ID NO: 3 - protein, 30 aa, homo sapiens
DCYCRIPACIAGERRYGTCIYQGRLWAFCC